SEQUENCE LISTING (20 sequences)

SEQ ID NO: 1 - protein, 13 aa, Artificial Sequence
  Description of Artificial Sequence Synthetic peptide
HGVSGHGQHGVHG

SEQ ID NO: 2 - protein, 12 aa, Artificial Sequence
  Description of Artificial Sequence Synthetic peptide
GVSGHGQHGVHG

SEQ ID NO: 3 - protein, 10 aa, Artificial Sequence
  Description of Artificial Sequence Synthetic peptide
VSGHGQHGVH

SEQ ID NO: 4 - protein, 8 aa, Artificial Sequence
  Description of Artificial Sequence Synthetic peptide
SGHGQHGV

SEQ ID NO: 5 - protein, 13 aa, Artificial Sequence
  Description of Artificial Sequence Synthetic peptide
PSLTGHGFHGVYD

SEQ ID NO: 6 - protein, 11 aa, Artificial Sequence
  Description of Artificial Sequence Synthetic peptide
FIVSAHGDHGV

SEQ ID NO: 7 - protein, 7 aa, Artificial Sequence
  Description of Artificial Sequence Synthetic peptide
THGQHGV

SEQ ID NO: 8 - protein, 7 aa, Artificial Sequence
  Description of Artificial Sequence Synthetic peptide
HGHGVHG

SEQ ID NO: 9 - protein, 10 aa, Artificial Sequence
  Description of Artificial Sequence Synthetic peptide
LASLHGQHGV

SEQ ID NO: 10 - protein, 13 aa, Artificial Sequence
  Description of Artificial Sequence Synthetic peptide
CVVTGHGSHGVFV

SEQ ID NO: 11 - protein, 10 aa, Artificial Sequence
  Description of Artificial Sequence Synthetic peptide
ISGHGQHGVP

SEQ ID NO: 12 - protein, 9 aa, Artificial Sequence
  Description of Artificial Sequence Synthetic peptide
CGHGNHGVH

SEQ ID NO: 13 - protein, 12 aa, Artificial Sequence
  Description of Artificial Sequence Synthetic peptide
IVARIHGQNHGV

SEQ ID NO: 14 - protein, 11 aa, Artificial Sequence
  Description of Artificial Sequence Synthetic peptide
HGSDGHGVQHG

SEQ ID NO: 15 - protein, 7 aa, Artificial Sequence
  Description of Artificial Sequence Synthetic peptide
FGHGHGV

SEQ ID NO: 16 - protein, 8 aa, Artificial Sequence
  Description of Artificial Sequence Synthetic peptide
HGNHGVLA

SEQ ID NO: 17 - protein, 12 aa, Artificial Sequence
  Description of Artificial Sequence Synthetic peptide
HGDSGHGQHGVD

SEQ ID NO: 18 - protein, 7 aa, Artificial Sequence
  Description of Artificial Sequence Synthetic peptide
HGHGVPL

SEQ ID NO: 19 - protein, 10 aa, Artificial Sequence
  Description of Artificial Sequence Synthetic peptide
SGHGAVHGVM

SEQ ID NO: 20 - protein, 12 aa, Artificial Sequence
  Description of Artificial Sequence Synthetic peptide
YAMSGHGHGVFI

(19) United States
(12) Patent Application Publication
(10) Pub. No.: US 2021/0106649 A1
(43) Pub. Date: Apr. 15, 2021

(54) SYNERGISTIC COMBINATION OF CHEMOTHERAPY AND PEPTIDE FOR TREATING CANCER

(71) Applicant: TOBEBIO Novel Drug Laboratory Co., Ltd., Suwon-si (KR)

(72) Inventors: Sun Jin KIM, Suwon-si (KR); Ho Jeong LEE, Seongnam-si (KR); Mira KIM, Seoul (KR); Youngeun HA, Hwaseong-si (KR); Gyeong-Yeon KIM, Hwaseong-si (KR); Gunny Cho, Seoul (KR)

(21) Appl. No.: 17/067,751

(22) Filed: Oct. 11, 2020

Related U.S. Application Data
(60) Provisional application No. 62/914,174, filed on Oct. 11, 2019.

(51) Int. Cl. A61K 38/10; A61K 9/00; A61K 31/337; A61K 33/243; A61K 31/282; A61P 35/00; A61K 31/513; A61K 31/52; A61K 31/7068; A61K 31/7072; A61K 31/7076; A61K 31/17

(52) U.S. Cl. CPC A61K 38/10; A61K 9/0019; A61K 31/337; A61K 33/243; A61K 31/282; A61K 31/17; A61K 31/513; A61K 31/52; A61K 31/7068; A61K 31/7072; A61K 31/7076; A61P 35/00

(57) ABSTRACT

The present invention relates to a method of treating cancer by administering alloferon in combination with a microtubule-stabilizing agent. The combination therapy provides improved therapeutic efficacy for treatment of solid tumors, including pancreatic cancer. Further provided herein includes a pharmaceutical composition for use in the combination therapy.

### 1. CROSS-REFERENCE TO RELATED APPLICATIONS

This application claims the benefit of U.S. Provisional Application No. 62/914,174, filed Oct. 11, 2019, which is hereby incorporated by reference in its entirety.

### 2. SEQUENCE LISTING

The instant application contains a Sequence Listing which has been submitted via EFS-Web and is hereby incorporated by reference in its entirety. Said ASCII copy, created on Dec. 14, 2020, is named 44465US_CRF_sequencelisting.txt and is 4.91 bytes in size.

### 3. BACKGROUND

Chemotherapy has long been the standard approach for treatment of cancers, together with surgery, radiation therapy, and more recently, immunotherapy. Chemotherapeutic agents commonly used for treating cancers include, but are not limited to, microtubule stabilizing agents (e.g., a taxane, such as paclitaxel, Nab-paclitaxel, docetaxel, or a modification thereof), platinum based agents (e.g., cisplatin, oxaliplatin, or carboplatin), alkylating agents (e.g., temozolomide), and antimetabolites (e.g., 5-fluorouracil (5-FU), 6-mercaptopurine (6-MP), capecitabine (Xeloda®), cytarabine (Ara-C®), floxuridine, fludarabine, gemcitabine (Gemzar®), or hydroxycarbamide).

Chemotherapy can be effective, but causes severe side effects, such as vomiting, low white blood cells (WBC), loss of hair, loss of weight and other toxic effects. Because of the extremely toxic side effects, many cancer patients cannot complete the intended regimen, and are thus unable to obtain the most effective therapeutic benefit. Adverse side effects associated with chemotherapeutic agents are generally the major dose-limiting toxicity (DLT) in the administration of these drugs. In addition, chemotherapy-induced side effects significantly impact the quality of life of the individual and may dramatically influence individual compliance with treatment.

For example, paclitaxel has been shown to have significant antineoplastic and anticancer effects in drug-refractory ovarian cancer, pancreatic cancer, and other cancer models. However, early development of paclitaxel was hampered by significant toxicities such as neutropenia and infection at clinically tolerable doses. An albumin formulation of paclitaxel, nab-paclitaxel (Abraxane), did achieve a statistical and clinically meaningful survival improvement for patients with various cancers, and has been approved by FDA for treatment of breast cancer, pancreatic cancer, and lung cancer. However, bone marrow suppression, primarily neutropenia, is still a dose-limiting toxicity of Abraxane. In clinical studies, Grade 3-4 neutropenia occurred in 34% of patients with metastatic breast cancer (MBC), 47% of patients with non-small cell lung cancer (NSCLC), and 38% of patients with pancreatic cancer.

To minimize such severe side effects, low-dose chemotherapy has been suggested as a new strategy for treatment of cancer. However, there has been a controversy as to whether low-dose chemotherapy can provide the desired therapeutic effects for treatment of cancer. Additionally, chemotherapy does not always work, and even when it is useful, it may not destroy the cancer completely. Therefore, cancer cells may persist in the body and often cause recurrence or metastasis. Although survival rates widely vary depending on cancer types and stages, the five-year survival rate for all stages of pancreatic cancer remains as low as 7% according to the American Cancer Society.

Accordingly, there has been a need for a new and improved chemotherapy for more safe and effective treatment of cancer.

### 4. SUMMARY

The present invention is based on a novel finding that therapeutic effects of a microtubule-stabilizing agent can be enhanced by adjunctively administering an effective amount of a pharmaceutical composition comprising a peptide called alloferon. Specifically, the present disclosure provides experimental data demonstrating that antitumor effects of a microtubule-stabilizing agent were significantly greater when administered in combination with alloferon-1, the peptide of SEQ ID NO: 1 (His-Gly-Val-Ser-Gly-His-Gly-Gln-His-Gly-Val-His-Gly), compared to when the microtubule-stabilizing agent or alloferon-1 was administered individually. Thus, the present invention provides an improved method of treating a cancer patient.

Accordingly, in one aspect, the present invention provides, in a method of treating a cancer patient with a microtubule-stabilizing agent, the improvement comprising: adjunctively administering to the cancer patient an effective amount of a pharmaceutical composition comprising the peptide of SEQ ID NO:1.

In some embodiments, the cancer patient has a solid tumor. In some embodiments, the patient has pancreatic cancer. In some embodiments, the subject has metastatic pancreatic cancer. In some embodiments, the subject has non-metastatic pancreatic cancer.

In some embodiments, the microtubule-stabilizing agent is a taxane. In some embodiments, the microtubule-stabilizing agent is paclitaxel, docetaxel, or a modification thereof. In some embodiments, the microtubule-stabilizing agent is paclitaxel or Nab-paclitaxel.

In some embodiments, the peptide pharmaceutical composition is administered once a day, twice a day, every other day, every three days, or once a week. In some embodiments, the pharmaceutical composition is administered at a peptide dose between 6 mg/m² and 75 mg/m². In some embodiments, the pharmaceutical composition is administered at a peptide dose from 10 to 50 mg/m², or from 20 to 40 mg/m².

In some embodiments, the peptide pharmaceutical composition is administered by s.c. injection.

Another aspect of the present disclosure provides a method of treating pancreatic cancer, comprising the steps of: administering to a subject with pancreatic cancer a first pharmaceutical composition comprising the peptide of SEQ ID NO:1, and administering to the subject a second pharmaceutical composition comprising a microtubule-stabilizing agent.

In some embodiments, the subject has metastatic pancreatic cancer. In some embodiments, the subject has non-metastatic pancreatic cancer.

In some embodiments, the microtubule-stabilizing agent is a taxane. In some embodiments, the microtubule-stabilizing agent is paclitaxel, docetaxel, or a modification thereof. In some embodiments, the microtubule-stabilizing agent is paclitaxel or Nab-paclitaxel.

In some embodiments, the microtubule-stabilizing agent is paclitaxel and administered at a dose between 100 mg/m² and 175 mg/m². In some embodiments, paclitaxel is administered every week, every 2-3 weeks, every 3-4 weeks, or every 4-5 weeks.

In some embodiments, the microtubule-stabilizing agent is Nab-paclitaxel and administered at a dose between 75 mg/m² and 125 mg/m². In some embodiments, Nab-paclitaxel is administered every week, every 2 weeks, every 3 weeks, every 4 weeks, every 2-3 weeks, every 3-4 weeks, or every 4-5 weeks.

In some embodiments, the microtubule-stabilizing agent is docetaxel and administered at a dose between 60 mg/m² and 100 mg/m². In some embodiments, docetaxel is administered every week, every 2 weeks, every 3 weeks, every 4 weeks, every 2-3 weeks, every 3-4 weeks, or every 4-5 weeks.

In some embodiments, the method further comprises administering a platinum-based agent. In some embodiments, the platinum-based agent is selected from the group consisting of cisplatin, oxaliplatin, and carboplatin. In some embodiments, the platinum-based agent is cisplatin.

In some embodiments, the method further comprises administering an antimetabolite. In some embodiments, the antimetabolite is selected from the group consisting of 5-fluorouracil, 6-mercaptopurine, capecitabine, cytarabine, floxuridine, fludarabine, gemcitabine, and hydroxycarbamide. In some embodiments, the antimetabolite is gemcitabine.

In some embodiments, the first pharmaceutical composition is administered once a day, twice a day, every other day, every three days, or once a week. In some embodiments, the second pharmaceutical composition is administered weekly, bi-weekly, once every three weeks, or once every four weeks.

In some embodiments, the first pharmaceutical composition is administered at a peptide dose between 6 mg/m² and 75 mg/m². In some embodiments, the first pharmaceutical composition is administered by s.c. injection.

In another aspect, the present disclosure provides a pharmaceutical composition in a unit dose, comprising the peptide of SEQ ID NO:1; and an excipient, wherein the unit dose includes the peptide at a dose between 1 mg and 150 mg. In some embodiments, the unit dose includes the peptide at a dose between 5 mg and 100 mg, between 10 mg and 100 mg, between 25 mg and 75 mg, or between 30 mg and 60 mg.

In yet another aspect, the present disclosure provides a kit for treating a subject with cancer comprising: a first pharmaceutical composition comprising the peptide of SEQ ID NO:1; and a second pharmaceutical composition comprising a microtubule-stabilizing agent.

In some embodiments, the microtubule-stabilizing agent is a taxane. In some embodiments, the microtubule-stabilizing agent is paclitaxel, docetaxel, or a modification thereof. In some embodiments, the microtubule-stabilizing agent is paclitaxel or Nab-paclitaxel.

In some embodiments, the kit further comprises a third pharmaceutical composition comprising a platinum-based agent. In some embodiments, the platinum-based agent is selected from the group consisting of cisplatin, oxaliplatin, and carboplatin. In some embodiments, the platinum-based agent is cisplatin.

In some embodiments, the kit further comprises a third pharmaceutical composition comprising an antimetabolite. In some embodiments, the antimetabolite is selected from the group consisting of 5-fluorouracil, 6-mercaptopurine, capecitabine, cytarabine, floxuridine, fludarabine, gemcitabine, and hydroxycarbamide. In some embodiments, the antimetabolite is gemcitabine.

In some embodiments, the first pharmaceutical composition is for administration once a day, twice a day, every other day, every three days or once a week. In some embodiments, the first pharmaceutical composition is in a unit dose. In some embodiments, the unit dose includes the peptide at a dose between 1 mg and 150 mg. In some embodiments, the first pharmaceutical composition is in an auto-injection pen. In some embodiments, the first pharmaceutical composition is a lyophilized powder.

In some embodiments, the second pharmaceutical composition is for weekly administration, bi-weekly administration, once in three-week administration, or once in four-week administration.

In one aspect, the present disclosure provides a peptide-containing pharmaceutical composition for use in a method of treating a cancer patient, the method comprising the steps of: administering to the cancer patient a pharmaceutical composition comprising a peptide of SEQ ID NO:1, and administering to the cancer patient a second pharmaceutical composition comprising a microtubule-stabilizing agent.

In some embodiments, the microtubule-stabilizing agent is a taxane. In some embodiments, the microtubule-stabilizing agent is paclitaxel, docetaxel, or a modification thereof. In some embodiments, the microtubule-stabilizing agent is paclitaxel or Nab-paclitaxel.

In some embodiments, the method further comprises administering a platinum-based agent. In some embodiments, the platinum-based agent is selected from the group consisting of cisplatin, oxaliplatin, or carboplatin. In some embodiments, the platinum-based agent is cisplatin.

In some embodiments, the method further comprises administering an antimetabolite. In some embodiments, the antimetabolite is selected from the group consisting of 5-fluorouracil, 6-mercaptopurine, capecitabine, cytarabine, floxuridine, fludarabine, gemcitabine, and hydroxycarbamide. In some embodiments, the antimetabolite is gemcitabine.

In some embodiments, the first pharmaceutical composition is for once a day, twice a day, every other day, every three days, or once a week administration. In some embodiments, the first pharmaceutical composition is in a unit dose. In some embodiments, the unit dose includes the peptide at a dose between 1 mg and 150 mg. In some embodiments, the first pharmaceutical composition is in an auto-injection pen. In some embodiments, the first pharmaceutical composition is a lyophilized powder.

In some embodiments, the second pharmaceutical composition is for weekly administration, bi-weekly administration, once in three-week administration, or once in four-week administration.

In some embodiments, the cancer patient has a solid tumor. In some embodiments, the cancer patient has pancreatic cancer. In some embodiments, the cancer patient has metastatic pancreatic cancer. In some embodiments, the cancer patient has non-metastatic pancreatic cancer.

### 5. BRIEF DESCRIPTION OF THE DRAWINGS

FIG. 1 shows in vivo bioluminescent imaging (BLI) intensity measured in mice orthotopically implanted with AsPC-1 pancreatic cancer cells and then treated with (a) control, (b) alloferon-1 alone, (c) paclitaxel alone, or (d) alloferon-1 together with paclitaxel.

FIG. 2 provides median survival rates of mice orthotopically implanted with AsPC-1 pancreatic cancer cells and then treated with (a) control, (b) alloferon-1 alone, (c) paclitaxel alone, or (d) alloferon-1 together with paclitaxel.

FIG. 3 provides body weight changes (%) over time in mice orthotopically implanted with AsPC-1 pancreatic cancer cells and then treated with (a) control, (b) alloferon-1 alone, (c) paclitaxel alone, or (d) alloferon-1 together with paclitaxel.

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

The term “effective amount” or “therapeutically effective amount” means an amount sufficient to produce a desired effect, e.g., an amount sufficient to reduce tumor burden or reduce disease or stabilize disease or reduce disease symptoms in a subject or an amount that is effective to ameliorate a symptom of a disease.

The terms “adjunctive administration” or “adjunctively administering” means administering a second therapeutic agent in sufficient temporal proximity to a first therapeutic agent to provide an additive or synergistic effect, or administering a first therapeutic agent in sufficient temporal proximity to a second therapeutic agent to provide an additive or synergistic effect. Adjunctive administration includes administration of the second therapeutic agent concurrent with (at the same time), sequential to (at a different time but on the same day, e.g., during the same patient visit), or separate from (on a different day) administration of a first therapeutic agent. For example, adjunctive administration of a peptide pharmaceutical composition in the present disclosure refers to administration of the peptide pharmaceutical composition in sufficient temporal proximity to administration of a chemotherapeutic agent to provide an additive or synergistic effect. Adjunctive administration of a peptide pharmaceutical composition may be concurrent with (at the same time), sequential to (at a different time but on the same day, e.g., during the same patient visit), or separate from (on a different day) administration of a chemotherapeutic agent.

The term “peptide pharmaceutical composition” as used herein refers to a pharmaceutical composition comprising a peptide. In preferred embodiments, the peptide pharmaceutical composition comprises the peptide of SEQ ID NO: 1 (“alloferon-1”).

The term “alloferon” as used herein refers to a peptide selected from the peptide group consisting of alloferon-1, alloferon-2, alloferon-3, alloferon-4, alloferon-5, alloferon-6, alloferon-7, alloferon-8, alloferon-9, alloferon-10, alloferon-11, alloferon-12, alloferon-13, alloferon-14, alloferon-15, alloferon-16, alloferon-17, alloferon-18, alloferon-19 and alloferon-20 as provided below in TABLE 1. Alloferon-1 refers to an alloferon with the amino acid sequence of SEQ ID NO: 1.

The term “treating cancer” as used herein, specifically refers to administering therapeutic agents to a patient diagnosed with cancer, i.e., having established cancer in the patient, to inhibit or to reduce the further growth or spread of the malignant cells in the cancerous tissue and/or to cause the death of malignant cells, or a patient in whom a cancer has been previously treated with potentially curative surgery, radiation, or other treatments and in whom the goal of treatment is to reduce the risk of cancer recurrence, or a patient at known high risk of developing a new cancer for whom the goal is cancer prevention.

The term “chemotherapy” or “chemotherapeutic agent” as used herein, refers to any chemical substances used in the art for the treatment of cancer and/or cancer-related conditions. Examples of chemotherapeutic agents include, but are not limited to, microtubule stabilizing agents (e.g., a taxane, such as paclitaxel, Nab-paclitaxel, docetaxel, or a modification thereof), platinum based therapy (e.g., cisplatin, oxaliplatin, or carboplatin), alkylating agents (e.g., temozolomide), antimetabolites (e.g., 5-fluorouracil (5-FU), 6-mercaptopurigne (6-MP), capecitabine (Xeloda®), cytarabine (Ara-C®), floxuridine, fludarabine, gemcitabine (Gemzar®), or Hydroxycarbamide), nucleoside analogues (e.g., 5-fluorouracil and capecitabine), topoisomerase inhibitors, hypomethylating agents, proteasome inhibitors, epipodophyllotoxins, DNA synthesis inhibitors, vinca alkaloids, or any combination thereof.

The term “analog” or “analog drug” as used herein refers to a drug presenting chemical and pharmacological similarity. An analog drug has a chemical structure similar to the corresponding drug.

The term “sufficient amount” as used herein refers to an amount sufficient to produce a desired effect. The amount can be an amount sufficient to produce desired effect by itself or in combination with another therapeutic agent.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50

### 6.3. Methods of Treating Cancer

In one aspect, an improved method of treating a cancer patient with a microtubule-stabilizing agent is provided. The improvement comprises: adjunctively administering to the cancer patient receiving a microtubule-stabilizing agent an effective amount of a pharmaceutical composition comprising the peptide of SEQ ID NO: 1 (alloferon-1). The present disclosure further provides a method of treating a cancer comprising the steps of administering to a subject with pancreatic cancer a first pharmaceutical composition comprising the peptide of SEQ ID NO: 1 (alloferon-1), and administering to the subject a second pharmaceutical composition comprising a microtubule-stabilizing agent.

Cancer Patients

The therapeutic methods provided herein are for treating cancer patients, particularly patients having solid tumors. In certain embodiments, the cancer is selected from the group consisting of: bladder cancer, breast cancer, cervical cancer, colorectal cancer, endometrial cancer, kidney cancer, lip and oral cancer, liver cancer, melanoma, mesothelioma, lung cancer, skin cancer, oral cancer, ovarian cancer, pancreatic cancer, prostate cancer, sarcoma, and thyroid cancer.

In preferred embodiments, the subject has a cancer of a type for which treatment with a microtubule-stabilizing agent is currently recommended and/or approved.

In some embodiments, the subject has a cancer of a type for which treatment with a microtubule-stabilizing agent is recommended and/or approved. For example, paclitaxel is currently recommended for treatment of ovarian cancer, breast cancer, lung cancer, Kaposi sarcoma, cervical cancer, or pancreatic cancer. Nab-paclitaxel is recommended for treatment of breast cancer, locally advanced or metastatic non-small cell lung cancer, or metastatic adenocarcinoma of the pancreas. Docetaxel is recommended for treatment of breast cancer, head and neck cancer, stomach cancer, prostate cancer or non-small-cell lung cancer. Nab-paclitaxel in combination with gemcitabine is recommended for treatment of pancreatic cancer.

In some embodiments, the subject has a cancer of a type for which treatment with a microtubule-stabilizing agent is recommended in combination with a platinum-based agent and/or an antimetabolite. In some embodiments, the subject has a cancer of a type for which treatment with paclitaxel in combination with gemcitabine is recommended. In some embodiments, the subject has a cancer of a type for which treatment with paclitaxel in combination with cisplatin is recommended. In some embodiments, the subject has a cancer of a type for which treatment with paclitaxel in combination with gemcitabine and cisplatin is recommended.

In some embodiments, the subject has been treated with a chemotherapeutic agent prior to initiating treatment with the combination therapy described herein. In some embodiments, the subject has never been treated with a chemotherapeutic agent prior to the combination therapy described herein.

In some embodiments, the subject has been treated with a microtubule-stabilizing agent prior to initiating treatment with the combination therapy described herein. In some embodiments, the subject has never been treated with a microtubule-stabilizing agent prior to the combination therapy described herein.

In certain embodiments, the subject has pancreatic cancer. In one embodiment, the subject has metastatic pancreatic cancer (MPC). In another embodiment, the subject has non-metastatic pancreatic cancer. In some embodiments, the subject has locally advanced pancreatic cancer (LAPC). In some embodiments, the subject has adenocarcinoma.

Suitable subjects for treatment also include subjects suffering from a disease or condition for which the recommended treatment regimen is treatment with a microtubule-stabilizing agent that has s side effect.

Alloferons

Alloferons are group of peptides originally isolated from insects and have been demonstrated to be capable of stimulating mouse and human NK cell cytotoxicity towards cancer cells. Alloferons used in the methods provided herein can be chemically or biologically synthesized. In some embodiments, alloferons are isolated and purified from natural products.

Examples of alloferons useful in the methods described herein are provided below in TABLE 1.

TABLE 1

| | Amino acid sequences of alloferons, (SEQ ID NOS 1-21) | | | | | | | | | | | | | | | |
|---|---|---|---|---|---|---|---|---|---|---|---|---|---|---|---|---|
| | Position | | | | | | | | | | | | | | | |
| Peptide | 1 | 2 | 3 | 4 | 5 | 6 | 7 | 8 | 9 | 10 | 11 | 12 | 13 | 14 | 15 | 16 |
| Alloferon- | His | Gly | Val | Ser | Gly | His | Gly | — | Gln | — | His | Gly | Val | His | Gly | — |
| 1 (SEQ | | | | | | | | | | | | | | | | |
| ID NO: 1) | | | | | | | | | | | | | | | | |
| Alloferon- | — | Gly | Val | Ser | Gly | His | Gly | — | Gln | — | His | Gly | Val | His | Gly | — |
| 2 (SEQ | | | | | | | | | | | | | | | | |
| ID NO: 2) | | | | | | | | | | | | | | | | |
| Alloferon- | — | — | Val | Ser | Gly | His | Gly | — | Gln | — | His | Gly | Val | His | — | — |
| 3 (SEQ | | | | | | | | | | | | | | | | |
| ID NO: 3) | | | | | | | | | | | | | | | | |
| Alloferon- | — | — | — | Ser | Gly | His | Gly | — | Gln | — | His | Gly | Val | | | — |
| 4 (SEQ | | | | | | | | | | | | | | | | |
| ID NO: 4) | | | | | | | | | | | | | | | | |
| Alloferon- | Pro | Ser | Leu | Thr | Gly | His | Gly | — | Phe | — | His | Gly | Val | Tyr | Asp | — |
| 5 (SEQ | | | | | | | | | | | | | | | | |
| IDNO: 5) | | | | | | | | | | | | | | | | |
| Alloferon- | Phe | Ile | Val | Ser | Ala | His | Gly | — | Asp | — | His | Gly | Val | — | — | — |
| 6 (SEQ | | | | | | | | | | | | | | | | |
| ID NO: 6) | | | | | | | | | | | | | | | | |
| Alloferon- | — | — | — | — | Thr | His | Gly | — | Gln | — | His | Gly | Val | — | — | — |
| 7 (SEQ | | | | | | | | | | | | | | | | |
| ID NO: 7) | | | | | | | | | | | | | | | | |
| Alloferon- | — | — | — | — | — | His | Gly | — | — | — | His | Gly | Val | His | Gly | — |
| 8 (SEQ | | | | | | | | | | | | | | | | |
| ID NO: 8) | | | | | | | | | | | | | | | | |
| Alloferon- | — | Leu | Ala | Ser | Leu | His | Gly | — | Gln | — | His | Gly | Val | — | — | — |
| 9 (SEQ | | | | | | | | | | | | | | | | |
| ID NO: 9) | | | | | | | | | | | | | | | | |
| Alloferon- | Cys | Val | Val | Thr | Gly | His | Gly | — | Ser | — | His | Gly | Val | Phe | Val | — |
| 10 (SEQ | | | | | | | | | | | | | | | | |
| ID NO: 10) | | | | | | | | | | | | | | | | |
| Alloferon- | — | — | Ile | Ser | Gly | His | Gly | — | Gln | — | His | Gly | Val | Pro | — | — |
| 11 (SEQ | | | | | | | | | | | | | | | | |
| ID NO: 11) | | | | | | | | | | | | | | | | |
| Alloferon- | — | — | — | Cys | Gly | His | Gly | — | Asn | — | His | Gly | Val | His | — | — |
| 12 (SEQ | | | | | | | | | | | | | | | | |
| ID NO: 12) | | | | | | | | | | | | | | | | |
| Alloferon- | Ile | Val | Ala | Arg | Ile | His | Gly | — | Gln | Asn | His | Gly | Val | — | — | — |
| 13 (SEQ | | | | | | | | | | | | | | | | |
| ID NO: 13) | | | | | | | | | | | | | | | | |
| Alloferon- | — | — | — | — | — | His | Gly | Ser | Asp | Gly | His | Gly | Val | Gln | His | Gly |
| 14 (SEQ | | | | | | | | | | | | | | | | |
| ID NO: 14) | | | | | | | | | | | | | | | | |
| Alloferon- | — | — | — | Phe | Gly | His | Gly | — | — | — | His | Gly | Val | — | — | — |
| 15 (SEQ | | | | | | | | | | | | | | | | |
| ID NO: 15) | | | | | | | | | | | | | | | | |
| Alloferon- | — | — | — | — | — | His | Gly | — | Asn | — | His | Gly | Val | Leu | Ala | — |
| 16 (SEQ | | | | | | | | | | | | | | | | |
| ID NO: 16) | | | | | | | | | | | | | | | | |
| Alloferon- | His | Gly | Asp | Ser | Gly | His | Gly | — | Gln | — | His | Gly | Val | Asp | — | — |
| 17 (SEQ | | | | | | | | | | | | | | | | |
| ID NO: 17) | | | | | | | | | | | | | | | | |
| Alloferon- | — | — | — | — | — | His | Gly | — | — | — | His | Gly | Val | Pro | Leu | — |
| 18 (SEQ | | | | | | | | | | | | | | | | |
| ID NO: 18) | | | | | | | | | | | | | | | | |
| Alloferon- | — | — | — | Ser | Gly | His | Gly | — | Ala | Val | His | Gly | Val | Met | — | — |
| 19 (SEQ | | | | | | | | | | | | | | | | |
| ID NO: 19) | | | | | | | | | | | | | | | | |
| Alloferon- | Tyr | Ala | Met | Ser | Gly | His | Gly | — | — | — | His | Gly | Val | Phe | Ile | — |
| 20 (SEQ ID | | | | | | | | | | | | | | | | |
| NO: 20) | | | | | | | | | | | | | | | | |

Alloferon-1, SEQ ID NO: 1, is used for various embodiments in the present disclosure. In some embodiments, a variant of alloferon-1, e.g., a peptide selected from alloferon 2-20, can be used.

In some embodiments, an alloferon mimetic is used. In some embodiments, the alloferon mimetic is an alloferon analog having a longer half-life in vivo as compared to alloferon. In some embodiments, the alloferon analog comprises a sequence selected from SEQ ID NO: 1-20.

In some embodiments, the alloferon mimetic is a conjugate of alloferon or an alloferon analog to a conjugate moiety. In some embodiments, the conjugate moiety is selected from polyethylene glycol (PEG) and hyaluronic acid. In some embodiments, the conjugate moiety is selected from the group consisting of HAS, human IgG, scFv, transferrin, albumin, and an Fc domain of an immunoglobulin. In some embodiments, the conjugate moiety is selected from the group consisting of: XTEN, a proline-alanine-serine polymer (PAS), a homopolymer of glycine residues (HAP), a gelatin-like protein (GLP), a signal peptide and an elastin-like peptide (ELP).

In some embodiments, the alloferon mimetic comprises one or more modified or non-naturally occurring amino acids, selected from the group consisting of: a steric enantiomer (D isomer), a rare amino acid of plant origin, a non-naturally occurring amino acid or amino acid mimetic, or have been modified by any one or more modifications selected from acetylation, acylation, phosphorylation, dephosphorylation, glycosylation, myristollation, amidation, aspartic acid/asparagine hydroxylation, phosphopantethane attachment, methylation, methylthiolation, prensyl group attachment, intein N-/C-terminal splicing, ADP-ribosylation, bromination, citrullination, deamination, dihydroxylation, formylation, geranyl-geranilation, glycation, palmitoylation, α-methyl-amino acids, Ca-methyl amino acids, and Nα-methyl amino acids.

In some embodiments, the alloferon mimetic comprises an N-terminal modification with acetylation, biotin, dansyl, 2,4-dinitrophenyl, fluorescein, 7-methoxycoumarin acetic acid (Mca), or palmitic acid. In some embodiments, the alloferon mimetic comprises an internal modification with cyclization (disulfide bonds), cysteine carbamidomethylation (CAM), isotope labeling, phosphorylation, or spacer (e.g., PEGylation, amino hexanoic acid). In some embodiments, the alloferon mimetic comprises a C-terminal modification with amide (amidation).

In some embodiments, the alloferon mimetic is chemically-synthesized and comprises one or more non-peptide bonds. In some embodiments, the pharmaceutically acceptable salt of an alloferon mimetic, wherein the salt is hydrochloride, trihydrochloride, sulfate, mesylate, or tosylate.

Chemotherapeutic Agent

In the methods described herein, alloferon is added to treatment with one or more chemotherapeutic agents to generate improved therapeutic outcomes and/or permit dose reduction of the chemotherapeutic agents without diminution in efficacy, reducing toxic side effects of the chemotherapeutic agents. In preferred embodiments, a chemotherapeutic agent previously known to be effective in treating a solid tumor is selected.

In particular, the chemotherapeutic agent can be a microtubule-stabilizing agent.

In some embodiments, the chemotherapeutic agent is paclitaxel. Paclitaxel is a microtubule stabilizing agent used to treat a number of types of cancer, including ovarian cancer, breast cancer, lung cancer, bladder cancer, prostate cancer, melanoma, esophageal cancer, Kaposi sarcoma, cervical cancer, and pancreatic cancer.

In certain embodiments, the chemotherapeutic agent used in the method of the present disclosure is a variant of paclitaxel. Albumin-bound paclitaxel (trade name Abraxane, also called nab-paclitaxel) is an alternative formulation where paclitaxel is bound to albumin nanoparticles. Abraxane was approved by the FDA in January 2005 for the treatment of breast cancer and it has since been approved for locally advanced or metastatic non-small cell lung cancer and metastatic adenocarcinoma of the pancreas as well. Albumin-bound paclitaxel was further approved for treatment of pancreatic cancer in combination with gemcitabine. Thus, the chemotherapeutic agent that can be used in the method of the present disclosure can be albumin-bound paclitaxel administered in combination with gemcitabine.

In certain embodiments, the chemotherapeutic agent used in the method of the present disclosure is docetaxel. Docetaxel is sold under the brand name Taxotere among others, and is a used to treat various types of cancer, including breast cancer, head and neck cancer, stomach cancer, prostate cancer and non-small-cell lung cancer.

In some embodiments, a plurality of chemotherapeutic agents are used. In these embodiments, the method further comprises administration of an additional chemotherapeutic agent.

In some embodiments, the additional chemotherapeutic agent is a platinum-based agent. Among platinum-based agents, cisplatin, oxaliplatin or carboplatin can be used in the method of the present disclosure.

Cisplatin (trade name Platinol® and Platinol®-AQ) has been used for treatment of testicular, ovarian, bladder, head and neck, esophageal, small and non-small cell lung, breast, cervical, stomach and prostate cancers, Hodgkin's and non-Hodgkin's lymphomas, neuroblastoma, sarcomas, multiple myeloma, melanoma, and mesothelioma. Oxaliplatin (trade name Eloxatin) has been used for treatment of colorectal cancer. In some cases, oxaliplatin is used in combination with fluorouracil and folinic acid (leucovorin). Carboplatin, sold under the trade name Paraplatin among others, is a chemotherapy medication used to treat a number of forms of cancer, including ovarian cancer, lung cancer, head and neck cancer, brain cancer, and neuroblastoma.

In some embodiments, the additional chemotherapeutic agent is an antimetabolite. In various embodiments, the antimetabolite is selected from the group consisting of 5-fluorouracil (5-FU), 6-mercaptopurine (6-MP), capecitabine (e.g., Xeloda®), cytarabine (e.g., Ara-C®), floxuridine, fludarabine, gemcitabine (e.g., Gemzar®), and hydroxycarbamide. Fluorouracil (5-FU), sold under the brand name Adrucil among others, has been used for treatment of colon cancer, esophageal cancer, stomach cancer, pancreatic cancer, breast cancer, and cervical cancer. 6-Mercaptopurine (6-MP) sold under the brand name Purinethol among others, has been used for treatment of acute lymphocytic leukemia (ALL), and chronic myeloid leukemia (CML). Capecitabine, sold under the brand name Xeloda among others, has been used for treatment of breast cancer, gastric cancer and colorectal cancer. Cytarabine, also known as cytosine arabinoside (ara-C), has been used for treatment of acute myeloid leukemia (AML), acute lymphocytic leukemia (ALL), chronic myelogenous leukemia (CML), and non-Hodgkin's lymphoma. Floxuridine (also known as 5-fluorodeoxyuridine) has been used for treatment of colorectal cancer, kidney cancer, and stomach cancer. Fludarabine, sold under the brand name Fludara among others, has been used for treatment of chronic lymphocytic leukemia, non-Hodgkin's lymphoma, acute myeloid leukemia, and acute lymphocytic leukemia. Gemcitabine (Gemzar®), has been used for treatment of breast cancer, ovarian cancer, non-small cell lung cancer, pancreatic cancer, and bladder cancer. Hydroxycarbamide, also known as hydroxyurea, has been used for the treatment of cervical cancer. Methotrexate (MTX), formerly known as amethopterin, has been used for treatment of breast cancer, lung cancer, and osteosarcoma. Pemetrexed (brand name Alimta) has been used for treatment of pleural mesothelioma and non-small cell lung cancer.

In some embodiments, a plurality of chemotherapeutic agents from different mechanistic classes are used. For example, a microtubule-stabilizing agent is used in combination with a platinum-based agent. In some embodiments, a microtubule-stabilizing agent is used in combination with an antimetabolite. In some embodiments, a microtubule-stabilizing agent is used together with a platinum-based agent and an antimetabolite. In one embodiment, paclitaxel or Nab-paclitaxel is used in combination with gemcitabine. In one embodiment, paclitaxel or Nab-paclitaxel is used in combination with cisplatin. In one embodiment, paclitaxel or Nab-paclitaxel is used in combination with gemcitabine and cisplatin.

In some embodiments, a microtubule-stabilizing agent is not used together with gemcitabine. In some embodiments, a microtubule-stabilizing agent is not used together with an immunosuppressor. In some embodiments, the immunosuppressor is cyclophosphamide. In some embodiments, a microtubule-stabilizing agent is not used together with a topoisomerase inhibitor. In some embodiments, the topoisomerase inhibitor is doxorubicin. In some embodiments, a microtubule-stabilizing agent is not used together with a vinca alkaloid. In some embodiments, the vinca alkaloid is vincristine. In some embodiments, a microtubule-stabilizing agent is not used together with cyclophosphamide, doxorubicin and vincristine.

Administration Methods

The methods of the present disclosure comprise adjunctive administration of a pharmaceutical composition comprising the peptide of SEQ ID NO:1 (alloferon-1) to a patient who is being treated with chemotherapy. In other words, the peptide pharmaceutical composition is administered concurrent with (at the same time), sequential to (at a different time but on the same day, e.g., during the same patient visit), or separate from (on a different day) administration of a chemotherapeutic agent, in each case in sufficient temporal proximity to administration of the chemotherapeutic agent as to provide an additive or synergistic effect.

In some embodiments, the peptide pharmaceutical composition is administered during the period while a chemotherapeutic agent is being administered. In some embodiments, the peptide pharmaceutical composition starts being administered when a chemotherapeutic agent starts being administered. In some embodiments, the peptide pharmaceutical composition stops being administered when a chemotherapeutic agent stops being administered. In some embodiments, the peptide pharmaceutical composition starts being administered before starting administration of a chemotherapeutic agent. In some embodiments, the peptide pharmaceutical composition continues being administered after completion of a chemotherapy.

The peptide pharmaceutical composition is administered in a therapeutically effective amount. In the methods described herein, the therapeutically effective amount, or dose, of a peptide pharmaceutical composition is a dose of the peptide effective to treat cancer in the subject in combination with a chemotherapeutic agent. In some embodiments, the peptide pharmaceutical composition is administered at a peptide dose sufficient to enhance therapeutic effects of a chemotherapeutic agent. In some embodiments, the peptide pharmaceutical composition is administered at a peptide dose sufficient to provide desired therapeutic effects when administered with a reduced dose of a chemotherapeutic agent.

The peptide pharmaceutical composition can be administered at a peptide dose between 0.1 mg/m² and 100 mg/m². In some embodiments, the peptide pharmaceutical composition is administered at a peptide dose between 0.6 mg/m² and 100 mg/m². In some embodiments, the peptide pharmaceutical composition is administered at a peptide dose between 0.6 mg/m² and 75 mg/m². In some embodiments, the peptide pharmaceutical composition is administered at a peptide dose between 6 mg/m² and 75 mg/m². In some embodiments, the pharmaceutical composition is administered at a peptide dose between 10 and 50 mg/m², or 20 and 40 mg/m². In some embodiments, the peptide pharmaceutical composition is administered at a peptide dose between 0.6 mg and 200 mg, between 0.6 mg and 150 mg, between 0.6 mg and 120 mg, between 0.6 mg and 60 mg, between 5 mg and 100 mg, between 10 mg and 100 mg, between 25 mg and 75 mg, or between 30 mg and 60 mg. In some embodiments, the peptide dose is injected by a single injection. In some embodiments, the peptide dose is injected by multiple injections.

The peptide pharmaceutical composition can be administered once a day, twice a day, or three times a day. In some embodiments, the peptide pharmaceutical composition is administered once every two days, once every three days, once every four days, or once in a week.

In some embodiments, the peptide pharmaceutical composition is administered for one week, two weeks, three weeks, four weeks, two months, three months, four months, five months, six months, seven months, eight months, nine months, ten months, one year, or longer.

In currently preferred embodiments, the peptide pharmaceutical composition is administered by injection. The peptide pharmaceutical composition can be injected subcutaneously or intradermally. In some embodiments, the peptide pharmaceutical composition is administered by intravascular injection. In certain embodiments, the peptide pharmaceutical composition is administered by retrograde intravenous injection. The peptide can be administered by injection of a liquid pharmaceutical composition.

The methods provided herein can comprise the steps of administering to a subject a first pharmaceutical composition comprising the peptide of SEQ ID NO: 1 and administering to the subject a second pharmaceutical composition comprising a chemotherapeutic agent. The first and the second pharmaceutical compositions can be administered concurrently or sequentially. In some embodiments, the first and the second pharmaceutical composition are administered via different routes of administration. In some embodiments, the first and the second pharmaceutical composition are administered via the same route of administration. In some embodiments, administration of the first pharmaceutical composition and the second pharmaceutical composition is performed separately, at least a few minutes apart, a few hours apart, one day apart, two days apart, three days apart, or one week apart. In some embodiments, the step of administering the first pharmaceutical composition is performed before the step of administering the second pharmaceutical composition.

In some embodiments, the step of administering the first pharmaceutical composition, the step of administering the second pharmaceutical composition, or both are repeated. In some embodiments, the step is repeated twice, three times, four times, five times, six times, or more.

In some embodiments, administration of the first pharmaceutical composition and administration of the second pharmaceutical composition continue for a month, for two months, for three months, for four months, for five months, for six months, or for longer. In some embodiments, administration of the first pharmaceutical composition and administration of the second pharmaceutical composition continue for a year, for two years, for three years, or longer.

In some embodiments, the first pharmaceutical composition and the second pharmaceutical composition are administered at different frequencies. For example, the first pharmaceutical composition containing the peptide is administered daily and the second pharmaceutical composition containing a chemotherapeutic agent is administered once every two days, once every three days, once every week, once every two weeks, once every three weeks, once every four weeks, once every month, once every two months, once every three months, or once every four months. In some embodiments, the first pharmaceutical composition containing the peptide is administered once a day, twice a day, three times a day, once every two days, once every three days, or once every week, and the second pharmaceutical composition containing a chemotherapeutic agent is administered once every two days, once every three days, once every week, once every two weeks, once every three weeks, once every four weeks, once every month, once every two months, once every three months, or once every four months.

In some embodiments, the method further comprises the step of administering a third pharmaceutical composition comprising a chemotherapeutic agent which is different from the chemotherapeutic agent in the second pharmaceutical composition. In some embodiments, the method further comprises the step of administering a fourth pharmaceutical composition comprising a chemotherapeutic agent which is different from the chemotherapeutic agent in the second pharmaceutical composition and different from the chemotherapeutic agent in the third pharmaceutical composition.

In some embodiments, a chemotherapeutic agent is administered pursuant to administration methods used in the art. Specifically, a chemotherapeutic agent is administrated using the method of administration that has been used for treating of corresponding cancer. For example, in certain embodiments, the chemotherapeutic agent is paclitaxel and administered at a dose between 100 mg/m² and 175 mg/m². In the embodiments, paclitaxel can be administered every week, every 2-3 weeks, every 3-4 weeks, or every 4-5 weeks. In certain embodiments, the chemotherapeutic agent is Nab-paclitaxel and administered at a dose between 75 mg/m² and 125 mg/m². In the embodiments, Nab-paclitaxel can be administered every week, every 2 weeks, every 3 weeks, every 4 weeks, every 2-3 weeks, every 3-4 weeks, or every 4-5 weeks. In some embodiments, the patient administered with Nab-paclitaxel is further administered with gemcitabine. In certain embodiments, the chemotherapeutic agent is docetaxel and administered at a dose between 60 mg/m² and 100 mg/m². In the embodiments, docetaxel can be administered every week, every 2 weeks, every 3 weeks, every 4 weeks, every 2-3 weeks, every 3-4 weeks, or every 4-5 weeks.

When combined with adjunctive administration of the peptide pharmaceutical composition, the administration method (e.g., dose and frequency) of the chemotherapeutic agent can be adjusted to obtain the desired therapeutic outcome. For example, the dose and/or frequency of a chemotherapeutic agent can be reduced to avoid side effects while achieving the desired efficacy when administered in combination with the peptide pharmaceutical composition. In some embodiments, the dose and/or frequency of a chemotherapeutic agent can be increased when administered in combination with the peptide pharmaceutical composition. In some embodiments, a chemotherapeutic agent is administrated using the method of administration that has been used for treating of corresponding cancer.

For example, in certain embodiments, the chemotherapeutic agent is paclitaxel and administered at a dose from 100 mg/m² to 200 mg/m², from 50 mg/m² to 100 mg/m², from 50 mg/m² to 75 mg/m², or from 25 mg/m² to 50 mg/m². In the embodiments, paclitaxel can be administered every three days, every four days, every five days, every six days, every week, every 2-3 weeks, every 3-4 weeks, every 4-5 weeks. In certain embodiments, the chemotherapeutic agent is Nab-paclitaxel and administered at a dose from 25 mg/m² to 50 mg/m², from 50 mg/m² to 75 mg/m², from 75 mg/m² to 100 mg/m², or from 100 mg/m² to 200 mg/m². In the embodiments, Nab-paclitaxel can be administered every two days, every three days, every four days, every five days, every six days, every week, every 2 weeks, every 3 weeks, every 4 weeks, every 2-3 weeks, every 3-4 weeks, or every 4-5 weeks. In some embodiments, the patient administered with Nab-paclitaxel is further administered with gemcitabine. In certain embodiments, the chemotherapeutic agent is docetaxel and administered at a dose from 25 mg/m² to 50 mg/m², from 30 mg/m² to 60 mg/m², from 45 mg/m² to 75 mg/m², or from 75 mg/m² to 200 mg/m². In the embodiments, docetaxel can be administered every week, every 2 weeks, every 3 weeks, every 4 weeks, every 2-3 weeks, every 3-4 weeks, or every 4-5 weeks.

In vivo and/or in vitro assays may optionally be employed to help identify optimal dosage ranges for the peptide and the chemotherapeutic agent when the chemotherapeutic agent is combined with the peptide pharmaceutical composition. The precise dose to be employed will also depend on the route of administration, and the seriousness of the condition, and should be decided according to the judgment of the practitioner and each subject's circumstances. Effective doses may be extrapolated from dose-response curves derived from in vitro or animal model test systems.

According to the conventional techniques known to those skilled in the art, the peptide pharmaceutical composition can be formulated with pharmaceutically acceptable carriers and/or vehicles, and can conveniently be packaged in unit dose form and multi-dose form. Non-limiting examples of the formulations include, but are not limited to, a solution, a suspension or an emulsion in oil or aqueous medium, an extract, an elixir, a powder for reconstitution, a granule, a tablet and a capsule, and may further comprise a dispersion agent or a stabilizer.

### 6.4. Peptide Pharmaceutical Composition

Another aspect of the present invention relates to a pharmaceutical composition comprising the peptide of SEQ ID NO: 1 and an excipient. The peptide pharmaceutical composition is for treatment of a cancer patient in combination with a chemotherapeutic agent.

Pharmaceutical Compositions

In some embodiments, the peptide is present in a liquid composition at a concentration between 1 mg/ml and 200 mg/ml, between 10 mg/ml and 400 mg/ml, between 5 mg/ml and 200 mg/ml, between 5 mg/ml and 100 mg/ml, between 10 mg/ml and 100 mg/ml, between 25 mg/ml and 75 mg/ml, or between 30 mg/ml and 60 mg/ml.

In some embodiments, the peptide is present in a liquid composition at a concentration from 1 mg/ml to 500 mg/ml, from 1 mg/ml to 400 mg/ml, from 10 mg/ml to 400 mg/ml, from 5 mg/ml to 400 mg/ml, from 10 mg/ml to 300 mg/ml, from 5 mg/ml to 200 mg/ml, from 5 mg/ml to 100 mg/ml, from 10 mg/ml to 100 mg/ml, from 25 mg/ml to 75 mg/ml, or from 30 mg/ml to 60 mg/ml.

In some embodiments, the peptide is present in a lyophilized composition.

For intravenous, intramuscular, intradermal, or subcutaneous injection, the peptide can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

In some embodiments, aprotic, polar solvents, such as DMSO, are used to stabilize peptide formulations against both chemical and physical degradation. The aprotic, polar solvent can improve the overall stability of peptides in a wide range of formulation conditions, including high concentrations and elevated or non-refrigerated temperatures, thus making possible the long-term storage of such peptides at elevated or room temperature, as well as the delivery of such peptides in long-term devices that would not otherwise be feasible, such as pen style injection devices or pump style delivery devices.

In some embodiments, the peptide pharmaceutical composition further comprises another therapeutic agent. For example, the peptide pharmaceutical composition can further comprise another therapeutic agent effective in treating cancer, e.g., a chemotherapeutic agent.

Unit Dosage Forms

In various embodiments, the peptide pharmaceutical composition is provided in a unit dosage form.

In particular embodiments, the unit dose contains between 1 mg and 150 mg of the peptide. In some embodiments, the unit dose is between 5 mg and 140 mg, between 5 mg and 120 mg, between 5 mg and 100 mg, between 10 mg and 100 mg, between 25 mg and 100 mg, between 25 mg and 75 mg, or between 30 mg and 60 mg. In some embodiments, the unit dose is 5 mg, 10 mg, 20 mg, 30 mg, 50 mg, 60 mg, 70 mg, 80 mg, 90 mg, 100 mg, 110 mg, 120 mg, 130 mg, 140 mg, or 150 mg. In some embodiments, the unit dosage form contains the peptide at a dose between 5 mg and 140 mg, between 5 mg and 120 mg, between 5 mg and 100 mg, between 10 mg and 100 mg, between 25 mg and 100 mg, between 25 mg and 75 mg, or between 30 mg and 60 mg. In some embodiments, the unit dosage form contains the peptide at a dose of 5 mg, 10 mg, 20 mg, 30 mg, 50 mg, 60 mg, 70 mg, 80 mg, 90 mg, 100 mg, 110 mg, 120 mg, 130 mg, 140 mg, or 150 mg.

In some embodiments, the pharmaceutical composition in the unit dosage form is in liquid form. In various embodiments, the unit dosage form contains between 0.1 ml and 50 ml of the pharmaceutical composition. In some embodiments, the unit dosage form contains 0.25 ml, 0.5 ml, 1 ml, 2.5 ml, 5 ml, 7.5 ml, 10 ml, 25 ml, or 50 ml of pharmaceutical composition. In some embodiments, the unit dosage form is a vial containing 1-5 ml of the pharmaceutical composition in a liquid form.

In particular embodiments, the unit dosage form is a vial containing 0.5 ml, 1 ml, 1.5 ml, 2 ml or 5 ml of the peptide pharmaceutical composition suitable for subcutaneous, intradermal, or intramuscular administration.

In various embodiments, the unit dosage form is a preloaded syringe, auto-injector, or auto-inject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single-use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an auto-inject pen. The auto-inject pen comprises an auto-inject pen containing a pharmaceutical composition as described herein. In some embodiments, the auto-inject pen delivers a predetermined volume of pharmaceutical composition. In other embodiments, the auto-inject pen is configured to deliver a volume of pharmaceutical composition set by the user.

In various embodiments, the auto-inject pen contains about 0.1 mL to about 5.0 mL of the pharmaceutical composition. In specific embodiments, the auto-inject pen contains about 0.5 mL of the pharmaceutical composition. In particular embodiments, the auto-inject pen contains about 1.0 mL of the pharmaceutical composition. In other embodiments, the auto-inject pen contains about 5.0 mL of the pharmaceutical composition.

Lyophilized Peptide Pharmaceutical Composition

In some embodiments, the unit dosage form is a vial containing a lyophilized peptide pharmaceutical composition. The lyophilized formulation can be reconstituted prior to use.

In some embodiments, the peptide is formulated with certain excipients, e.g., a carbohydrate and a salt, prior to lyophilization. Stability of the peptide can be increased by formulating the peptide prior to lyophilization with an aqueous solution comprising a stabilizing agent. Compositions known to stabilize a peptide in lyophilized formulations can be used in various embodiments. For example, N-acetyl-L-cysteine, N-ethyl-maleimide, and/or cysteine have been used to stabilize proteins in liquid or lyophilized formulations without coupling to free thiols. This approach allowed the stabilization of the peptide having a free thiol in the liquid formulation prior to the start of the lyophilization process, and also in the lyophilized product by reducing or inhibiting the formation of the disulfide-linked aggregates.

In some embodiments, the peptide is lyophilized from a solution with a pH ranging from about pH 4.0 to about pH 7.5. In some embodiments, the peptide is lyophilized from a solution with a pH ranging from about pH 4.0 to about pH 6.0. In some embodiments, the peptide is lyophilized from a solution with a pH of about pH 4.5.

The final concentration of the peptide in liquid compositions reconstituted from lyophilized formulations can be between 10 mg/ml and 400 mg/ml, between 5 mg/ml and 200 mg/ml, between 5 mg/ml and 100 mg/ml, between 10 mg/ml and 100 mg/ml, between 25 mg/ml and 75 mg/ml, or between 30 mg/ml and 50 mg/ml.

In lyophilized embodiments, the peptide formulation is lyophilized under standard conditions known in the art. A method for lyophilization of the peptide formulation of the invention may comprise (a) loading a container (e.g., a vial), with a peptide formulation and an excipient, into a lyophilizer (b) cooling the peptide formulation to sub-zero temperatures; and (c) substantially drying the peptide formulation. The conditions for lyophilization, e.g., temperature and duration, of the peptide formulation of the invention can be adjusted by a person of ordinary skill in the art taking into consideration factors that affect lyophilization parameters, e.g., the type of lyophilization machine used, the amount of the peptide used, and the size of the container used.

The container holding the lyophilized peptide formulation may then be sealed and stored for an extended period of time at various temperatures (e.g., room temperature to about −180° C., preferably about 2-8° C. to about −80° C., more preferably about −20° C. to about −80° C., and most preferably about −20° C.). In certain aspects, the lyophilized peptide formulations are preferably stable within a range of from about 2-8° C. to about −80° C. for a period of at least 6 months without losing significant activity. Storage time may be as long as several months, 1 year, 5 years, or up to 10 years. Preferably the preparation is stable for a period of at least about 3 years.

### 6.5. Kits for Combination Therapy

In another aspect, the present invention provides a kit for a combination therapy of a subject with cancer. The kit can comprise a first pharmaceutical composition comprising the peptide of SEQ ID NO: 1 and a second pharmaceutical composition comprising a chemotherapeutic agent.

In some embodiments, the first pharmaceutical composition and the second pharmaceutical composition are in a single container. In some embodiments, the first pharmaceutical composition and the second pharmaceutical composition are separate pharmaceutical compositions in two or more separate containers.

The kit can comprise one or more unit doses of the first pharmaceutical composition. The kit can further comprise one or more unit doses of the second pharmaceutical composition. In some embodiments, the kit comprises one or more vials containing the first pharmaceutical composition, and one or more vials containing the second pharmaceutical composition.

The kit can further comprise an instruction explaining the method of administering the first pharmaceutical composition, the second pharmaceutical composition, or both. The method can be any of the administration methods provided herein.

### 6.6. Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art.

### Example 1: Experimental Methods for Testing Synergistic Effects of Chemotherapeutic Agent and Alloferon-1

### Preparation of Therapeutic Agents

Paclitaxel and other chemotherapeutic agents were obtained from commercial vendors. In the specific experiment described in Example 2, Taxol® (BMS, NSC number: 125973) was used as paclitaxel.

The synthesis of alloferon-1 (SEQ ID NO: 1) was accomplished according to the solid phase methodology described by Merrifield (J. Am. Chem. Soc. 85, 2149-2154 (1963)). The peptide was purified by reverse phase High Performance Liquid Chromatography (HPLC) and identified by mass spectrometry (MS). The peptide was supplied as a dry powder in salt form and was stored at −20° C. protected from light.

### Cell Cultures

AsPC-1, human pancreas adenocarcinoma cell line, was purchased from American Type Culture Collection (ATCC) and maintained in a complete Eagle's minimal essential medium (MEM) medium supplemented with 10% fetal bovine serum (FBS) (Hyclone), non-essential amino acid, sodium pyruvate, penicillin-streptomycin and vitamin solution (Life Technologies). AsPC-1 cells were incubated at 37° C. in a mixture atmosphere of 5% CO₂ and 95% O₂. Cell lines were authenticated via short tandem repeat (STR) profiling (Cosmogenetech).

### Establishment of Stable Luciferase-Labelled Cell Lines (AsPC-1-Luc)

AsPC-1 cells were seeded into 6 well plates at a density of 50-70% and incubated overnight. CMV-Firefly luciferase lentivirus (Cellomics) was diluted in a MEM medium containing 8 μg/mL polybrene (SigmaAldrich) and added to well. Cells were incubated overnight, and then medium was replaced with fresh MEM. The Stable clones were selected using 2 μg/mL puromycin (Life Technologies) and individual clones were screened for luciferase activity by measuring their light emission with the IVIS® Lumina III In vivo Imaging System (PerkinElmer) after application of D-luciferin (GoldBio).

### Establishment of Experimental Pancreatic Cancer Model in Nude Mice

6 weeks-old female athymic nude mice were purchased from the Orient. The mice were housed and maintained under pathogen-free conditions. Luciferase labeled AsPC-1 cells were harvested and washed with serum-free medium, and re-suspended at a final concentration of 1×10⁵ cells in 50 μL Ca²⁺/Mg²⁺-free Hank's balanced salt solution (HBSS). Mice were anesthetized by intraperitoneal injection with Avertin for surgery. The left abdominal flank skin and muscle of mice was incised, and the pancreatic lobes was visualized. The AsPC-1 cells suspended in HBSS were then directly injected into the pancreas. The muscle and skin layers incised were closed with wound clips (Clay Adams). Tumor growth was monitored weekly with IVIS® Lumina III In vivo Imaging System.

### Example 2: Synergistic Effects of Chemotherapy and Peptide in Cancer Mouse Model

Therapeutic effects of alloferon-1 in combination with a microtubule-stabilizing agent, paclitaxel, was tested in vivo in the cancer mouse model generated by orthotopic implantation of allografts or xenografts of pancreatic cancer cells. Before testing the agents, each of the mice was examined by bioluminescent imaging analysis using the IVIS Lumina III Imaging System to confirm that orthotopically implanted AsPC-1 pancreatic cancer cells were established (10-14 days after implantation).

Subject mice were divided into four separate groups, and each group was treated with (1) control (vehicle, saline), (2) alloferon-1 alone, (3) paclitaxel alone, or (4) alloferon-1 in combination with paclitaxel, respectively. Alloferon-1 obtained in powder form was dissolved in saline and then administered at a dose of 2.5 mg/kg daily by subcutaneous injection. Paclitaxel was administrated weekly by intraperitoneal injection at a dose of 8 mg/kg. The injections of alloferon-1 and paclitaxel continued throughout the survival of the subject mice. Throughout the course of the survival studies, bioluminescence image, body weight and clinical signs were monitored. When body weights decreased by more than 30% of the weights measured at the time of tumor implantation, the subject mice were euthanized and pancreatic tissues were harvested for histological analysis.

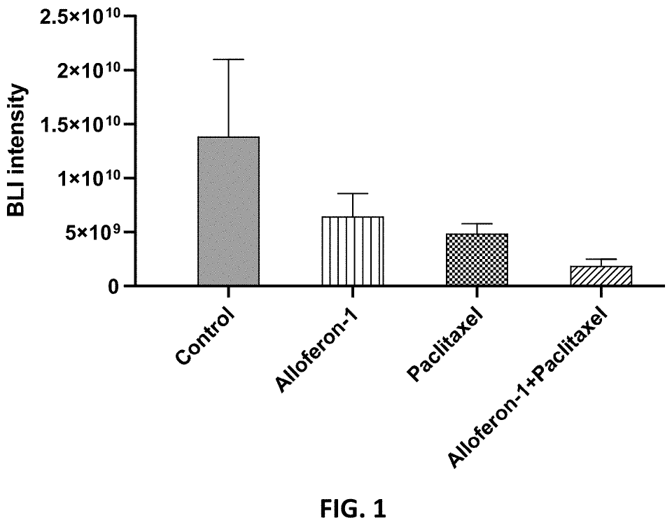

Tumor growth measured by bioluminescence imaging based on BLI intensity in each treatment group is summarized in FIG. 1. The results show that the combined administration of alloferon-1 and paclitaxel has significantly greater antitumor activities against the human pancreatic cancer AsPC-1, compared to control, alloferon-1 alone or paclitaxel alone.

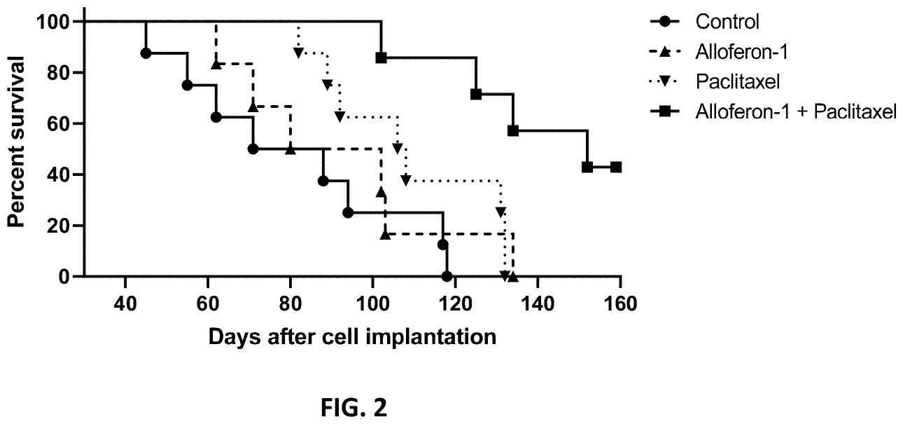

The number of survival days for all animals was recorded and statistical analysis was performed using the GraphPad Prism 8. Kaplan-Meier survival plots were analyzed with the Log-rank (Mantel-Cox) test to compare survival rates between groups. A probability (P) value of <0.05 or lower was considered statistically significant. Results obtained from the analysis are provided in FIG. 2 and TABLE 2. The results show that the mouse group treated with both alloferon-1 and paclitaxel had significantly greater median survival rates, compared mice treated with control, alloferon-1 alone, or paclitaxel alone. This demonstrates that combination therapy of alloferon-1 and paclitaxel elicited synergistic effect, in terms of median survival, against the human pancreatic cancer AsPC-1.

TABLE 2
Survival analysis of AsPC-1 Pancreatic Cancer Orthotopic
Mice Model

| | | | | | Allo- |
|---|---|---|---|---|---|
| | | | Allo- | Pacli- | feron-1 + |
| Groups | | Control | feron-1 | taxel | Paclitaxel |
| Number of total mice | | 8 | 6 | 8 | 7 |
| Median survival, Days | | 79.5 | 91 | 107 | 152 |
| Median | vs. Control | — | 0.477 | 0.059 | 0.0005 |
| survival | vs. Alloferon-1 | — | — | 0.5242 | 0.0072 |
| P-value | vs. Paclitaxel | — | — | — | 0.0088 |

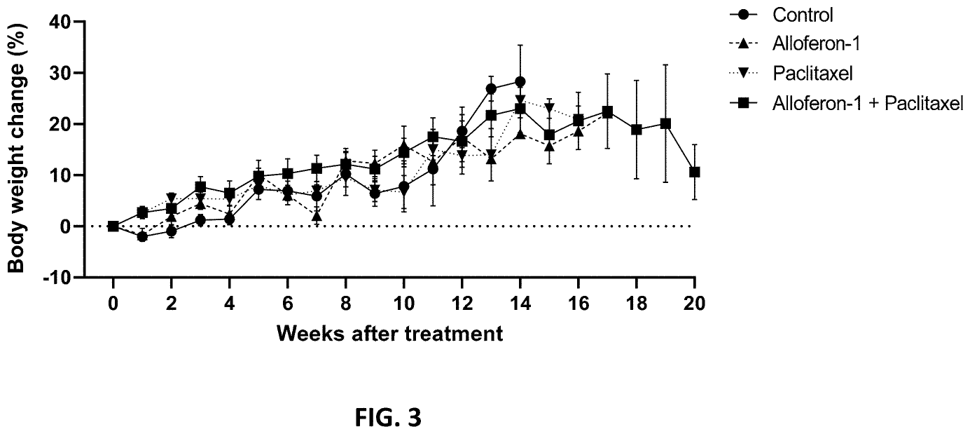

Body weights measured in each animal group throughout the study are also provided in FIG. 3. There was no significant different among the groups, suggesting that the combination therapy of alloferon-1 and paclitaxel does not cause toxicity, in terms of body weight change, against the tumor-bearing mice model.

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. In a method of treating a cancer patient with a microtubule-stabilizing agent, the improvement comprising:
adjunctively administering to the cancer patient an effective amount of a pharmaceutical composition comprising the peptide
(SEQ ID NO: l)
His-Gly-Val-Ser-Gly-His-Gly-Gln-His-Gly-Val-His-
Gly.

2. The method of claim 1, wherein the cancer patient has a solid tumor.

3. The method of claim 1, wherein the patient has pancreatic cancer, optionally wherein the pancreatic cancer is metastatic pancreatic cancer or non-metastatic pancreatic cancer.

4-5. (canceled)

6. The method of claim 1, wherein the microtubule-stabilizing agent is a taxane.

7. The method of claim 6, wherein the microtubule-stabilizing agent is paclitaxel, Nab-paclitaxel, docetaxel, or a modification thereof.

8. (canceled)

9. The method of claim 1, wherein the peptide pharmaceutical composition is administered once a day, twice a day, every other day, every three days, or once a week.

10. The method of claim 1, wherein the pharmaceutical composition is administered at a peptide dose from 6 mg/m² to 75 mg/m², from 10 to 50 mg/m², or from 20 to 40 mg/m².

11. (canceled)

12. The method of claim 1, wherein the peptide pharmaceutical composition is administered by s.c. injection.

13. A method of treating pancreatic cancer, comprising the steps of:
administering to a subject with pancreatic cancer an effective amount of a first pharmaceutical composition comprising the peptide
(SEQ ID NO: 1)
His-Gly-Val-Ser-Gly-His-Gly-Gln-His-Gly-Val-His-
Gly,
and
adjunctively administering to the subject an effective amount of a second pharmaceutical composition comprising a microtubule-stabilizing agent.

14. The method of claim 13, wherein the subject has metastatic pancreatic cancer or non-metastatic pancreatic cancer.

15. (canceled)

16. The method of claim 13, wherein the microtubule-stabilizing agent is a taxane, optionally selected from paclitaxel, docetaxel, Nab-paclitaxel, or a modification thereof.

17-24. (canceled)

25. The method of claim 13, further comprising administering a platinum-based agent.

26. The method of claim 25, wherein the platinum-based agent is selected from the group consisting of cisplatin, oxaliplatin, and carboplatin.

27. (canceled)

28. The method of claim 13, further comprising administering an antimetabolite.

29. The method of claim 28, wherein the antimetabolite is selected from the group consisting of 5-fluorouracil, 6-mercaptopurine, capecitabine, cytarabine, floxuridine, fludarabine, gemcitabine, and hydroxycarbamide.

30-32. (canceled)

33. The method of claim 13, wherein the first pharmaceutical composition is administered at a peptide dose between 6 mg/m² and 75 mg/m².

34. The method of claim 13, wherein the first pharmaceutical composition is administered by s.c. injection.

35. A pharmaceutical composition in a unit dose, comprising the peptide
(SEQ ID NO: 1)
His-Gly-Val-Ser-Gly-His-Gly-Gln-His-Gly-Val-His-
Gly;
and an excipient,
wherein the unit dose includes the peptide at a dose between 1 mg and 150 mg.

36. (canceled)

37. A kit for treating a subject with cancer comprising:
a first pharmaceutical composition comprising the peptide
His-Gly-Val-Ser-Gly-His-Gly-Gln-His-Gly-Val-His-Gly (SEQ ID NO:1); and
a second pharmaceutical composition comprising a microtubule-stabilizing agent.

38-72. (canceled)
